(19) United States
(12) Patent Application Publication
(10) Pub. No.: US 2009/0092231 A1
(43) Pub. Date: Apr. 9, 2009

(54) X-RAY DEVICE FOR PRODUCING X-RAY IMAGES

(76) Inventor: Franz Beimler, Weiden (DE)

Correspondence Address: BRINKS HOFER GILSON & LIONE, P.O. BOX 10395, CHICAGO, IL, 60610, US

(21) Appl. No.: 12/245,411

(22) Filed: Oct. 3, 2008

(30) Foreign Application Priority Data
Oct. 8, 2007 (DE) 10 2007 048 166.9

(51) Int. Cl. A61B 6/00; G21K 1/00

(52) U.S. Cl. 378/145; 378/19

(57) ABSTRACT

X-ray device for producing X-ray images is provided. The X-ray device includes a radiation generator unit embodied for the purpose of generating an X-ray field and an image receiver that has a larger receiving area compared to the area of the X-ray field that is to be generated or has been generated, wherein the radiation generator unit and the receiving area of the image receiver are embodied to be movable relative to one another.

This patent document claims the benefit of German Patent Application No. DE 10 2007 048 166.9, filed Oct. 8, 2007, which is hereby incorporated by reference.

### BACKGROUND

The present embodiments relate to an X-ray device for producing X-ray images, in particular for producing X-ray images in an end position of at least one movement axis of the X-ray device.

In the course of X-ray examinations in which it is necessary to produce images that lie in an end position of the movement axis or in a movement axis of an X-ray machine, in which an emitter/detector unit has to be moved to the end of a patient examination table, it was previously possible to take the images by using a radiation-limiting unit, such as a multi-leaf collimator. The radiation-limiting unit is used to mask-out an X-ray field that is generated, which is larger than that required for taking the actual image and extends to the edge of the image receiver area. However, this approach exposes the patient to an increased amount of radiation.

It is also possible to reposition the patient or to move the patient into a possibly uncomfortable position in which the region to be imaged is placed further toward the receiver center in order to acquire the desired image. However, a certain amount of effort is necessary to reposition the patient during an ongoing examination, while problems with regard to the comparability of images that were taken in different positions can arise. When the patient assumes an uncomfortable position, For example, has to bend over a positioning surface, the position can be painful and in certain cases even harmful.

There are attempts in certain applications in cardiology and neurology to use asymmetric grids in X-ray devices, by which grid units that permit an asymmetric masking-in, in relation to the central X-ray beam. The asymmetric grid units are complicated and expensive, for example, because the individual grids that form the grid pairs have to be moved separately via corresponding drive devices.

### SUMMARY AND DESCRIPTION

The present embodiments may obviate one or more of the drawbacks or limitations inherent in the related art. For example, in one embodiment, an X-ray device uses an improved method for producing X-ray images.

In one embodiment, an X-ray device includes a radiation generator unit that generates an X-ray field and an image receiver that has a larger receiving area compared to the area of the X-ray field that is to be generated or has been generated. The X-ray device produces X-ray images, such as X-ray images in an end position of at least one movement axis of the X-ray device. The radiation generator unit and the receiving area of the image receiver are movable relative to one another.

The radiation generator unit of the X-ray device and the receiving area of the image receiver of the device, for example, the surface area of a solid-state detector, are no longer of necessity moved coupled to one another. A relative movement between the emitter unit and the receiving area is made possible. The relative movement allows the edge zones of the receiving area to be irradiated in a targeted manner, such that images may be acquired in a simple manner, for example, in an end position of a movement axis of the X-ray device.

Owing to this possibility of a relative movement the X-ray field that is generated or illuminated on the receiver can be chosen precisely in the size actually required for acquiring the image or, as the case may be, a laborious repositioning or placing of the patient in an uncomfortable posture can be prevented. At the same time it is no longer necessary, when a relative movement is possible, to provide complex and expensive grid units that permit an asymmetric masking-in.

The radiation generator unit and the receiving area of the image receiver are movable relative to one another such that the receiving area of the image receiver can be masked out asymmetrically with respect to its center and/or the X-ray field that is to be generated or has been generated lies asymmetrically with respect to the center of the receiving area of the image receiver.

The X-ray field that is masked out or generated is not located around the center of the image receiver or receiving area. The X-ray field is asymmetrical with respect to the central point or center of, for example, a detector area. The radiation generator unit is movable relative to the receiving area of the image receiver that, for example, edge zones of the receiving area can be irradiated by the generated radiation, without the radiation field simultaneously needing to have an unnecessarily large extension.

Grids may be provided. The grids may be assigned to the radiation generator unit such that accordingly a suitably large asymmetric field is masked out. Grids do not have to be used, but instead an asymmetric X-ray field can be generated directly, for example, an X-ray field that is asymmetric with respect to the center of the receiving area of the image receiver. The masked-out area is to be understood as the area in which radiation strikes the receiving area of the image receiver.

The radiation generator unit and the receiving area of the image receiver can be movable relative to one another by rotating the radiation generator unit through an angle relative to the receiving area of the image receiver and/or by rotating the receiving area of the image receiver through an angle relative to the radiation generator unit.

The emitter unit and the receiving area of the image receiver are operable to be moved relative to each other. One of these two elements of the X-ray device, namely the emitter or the receiver, may be rotated or pivoted relative to the other element. The radiation generator unit may be rotated through a certain angle, for example, α, relative to the receiving area of the image receiver. Alternatively, instead of the radiation generator unit, the image receiver may be rotated, at least slightly. A corresponding rotatable mount may be used to rotate the image receiver.

By rotating the radiation generator unit or possibly the receiving area it is possible to irradiate edge zones of the receiving area, with the X-ray field being generated asymmetrically with respect to the receiving area on account of the underlying rotating or pivoting movement, with the result that the size can be adjusted. Accordingly, the patient may not be exposed to an unnecessarily high dose of radiation.

Alternatively, or in addition, the radiation generator unit and the receiving area of the image receiver may be movable relative to one another by displacing the radiation generator unit relative to the receiving area of the image receiver, for example, by displacing radiation generator unit parallel to the image receiver plane, and/or by displacing the receiving area of the image receiver relative to the radiation generator unit. The radiation generator unit may be displaced, while the image receiver remains in position (already assumed for the image acquisition).

If the emitter unit is displaced accordingly by a certain path relative to the image receiver unit, edge zones of the detector area can be illuminated. This can possibly remove the need for a laborious repositioning of the patient.

Rotating the emitter unit through an angle relative to the image receiver unit and of displacing the emitter unit through a certain path relative to the image receiver unit have the advantage that inexpensive, symmetrically structured grid units can be used for masking out the radiation, grid units in which the grid pairs are moved only symmetrically with respect to the central X-ray beam.

Displacing, for example, the emitter offers the advantage that the central beam of the masked-in X-ray field which is smaller than the possible radiation-sensitive receiving area or detector area of a solid-state detector strikes at right angles onto the object to be imaged and at right angles onto the image receiver, with the result that image distortions (due to striking at an oblique angle) are prevented.

The displacing may be performed in such a way that the distance of the emitter unit with respect to the receiving area or receiver plane remains unchanged. A parallel displacement relative to the detector plane or image receiver plane may be performed.

Sub-fields of the solid-state detector may be exposed to radiation with the aid of commercially available multi-leaf collimators. For example, in the direction of the longitudinal axis of the patient's body, thereby cost-effectively contributing toward reducing the medically necessary radiation dose to which a patient is exposed.

The image receiver may be a solid-state detector. Solid-state detectors enable large-area image recording in which a uniformly true-to-scale image is generated in all sub-fields of the detector. Solid-state detectors may be designed such that the incoming X-ray radiation is converted into visible light with the aid of a scintillator. Charge is then generated in turn from the light in a detector matrix. The charge may be stored and read out. As well as these indirect solid-state detectors there are direct solid-state detectors in which the incident photons generate charges which are extracted with the aid of electrodes.

Larger receiving areas are available which generally are only partially used. Thus in most cases, not least for reasons of radiation hygiene, only sub-fields of the detector are masked out, whereby as a result of the inventive asymmetric masking-out edge zones can now be used in a targeted manner for the image acquisition by the fields being generated in the direction of the edge zones.

A symmetrically structured grid unit may be assigned to the radiation generator unit for generating radiation. The grid unit includes of grid pairs which can only be moved jointly) symmetrically with respect to the central beam. This can be realized comparatively inexpensively and without particularly complex drive units.

The grid unit may be built using grid pairs that can be moved symmetrically with respect to the central beam. The possibility of movement enabling the generated radiation field to be reduced or increased in size. Expensive asymmetric grids are not necessary.

The radiation generator unit and the receiving area of the image receiver can be coupled to allow a relative movement by a releasable movement coupling device.

The movement of the radiation generator unit may be coupled to the image receiver. The radiation generator unit and the image receiver move together in the coupled state, though the coupling can be released so as to allow the relative movement. The coupling is ensured via a movement coupling device which can be released again according to the invention in order to cancel the coupling. It therefore continues to be possible to use the movement coupling for unproblematic image acquisition, for example, to move the emitter and receiver uniformly, whereas the coupling may be released for acquiring images in the end positions of the movement axis of the X-ray machine.

A mechanical and/or electromechanical movement coupling device may be provided as a releasable movement coupling device. The possible movement coupling devices may be based on different principles. Different types of coupling may be combined, for example, a mechanical coupling that has to be released manually, with an electromechanical coupling, which (e.g., for safety reasons) continues to exist after the mechanical coupling has already been released. The electromechanical coupling may be released. A more variable setting and a releasing via an operator action on a remote control device, for example, not directly on the machine are possible. One or another coupling mechanism will be used.

Following the releasing of the movement coupling device, the radiation generator unit and/or the receiving area of the image receiver may be movable and/or guidable relative to one another manually and/or in a motor-driven manner, for example, by moving the radiation generator unit parallel to the longitudinal axis of a positioning device of the X-ray device. It is therefore possible to carry out the relative movement manually after the decoupling or, as the case may be, in the case of an easily releasable coupling. The manual movement may be effected, for example, such that initially a certain coupling resistance is overcome in order to cancel the coupling. Apart from that or in addition, it is possible to generate the relative movement in a motor-driven manner. This offers the advantage that it is not necessary to approach the X-ray machine directly in order to generate the movement and a fine adjustment may be achieved with less probability of error more easily using automated devices. The radiation generator unit may be moved after the releasing of the movement parallel to the longitudinal axis of a positioning device for the patient that is provided as part of the X-ray device. The radiation generator unit is moved, for example, parallel to a longitudinal axis of a patient examination table on which the patient is placed for the purpose of taking X-ray images.

The radiation generator unit and the receiving area of the image receiver may be moved relative to one another continuously and/or incrementally. It is therefore possible where appropriate to implement an infinitely variable adjustability relative to one another, usually in the direction of a longitudinal axis of the X-ray device. The relative position may be adjusted incrementally. For example, the longitudinal axis may be adjusted in specific increments with respect to the centered position of the emitter/image receiver center.

A continuous and an incremental adjustability to be implemented for X-ray devices so that it is possible to switch or select between these two options, such that, for example, a stepwise movability is fundamentally provided, from which it is possible to switch to a continuous movability in order to enable a particularly fine adjustment, in the case of complex image acquisition processes, for example.

The movement axis of the emitter may be decouplable by a mechanical or other decoupling mechanism, for example, by manual actuation or manual guidance. An electromechanical (and therefore releasable) coupling mechanism may be used in which a variable motor-driven longitudinal axis adjustment is possible. Asymmetric radiation may be easily masked in upon decoupling.

A method for producing X-ray images is provided. The method may be performed using an X-ray device, such as an X-ray device as described in the foregoing. The method may produce X-ray images in an end position of at least one movement axis of the X-ray device, by a radiation generator unit embodied for the purpose of generating an X-ray field and an image receiver that has a larger receiving area compared to the area of the X-ray field that is to be generated or has been generated. The method is characterized in that the radiation generator unit and the receiving area of the image receiver are moved relative to one another in the course of producing the X-ray images.

In one embodiment, an X-ray field is generated with the aid of a radiation generator unit and wherein the image acquisition takes place using an image receiver whose receiving area is generally larger than the X-ray field that is typically generated. To generate an overview image, for example, an X-ray field is generated which is as large as the receiving area of the detector. For radiation hygiene reasons, a smaller section of the receiving area is masked out or irradiated.

In one embodiment, the radiation generator unit and the receiving area of the image receiver are moved relative to one another during the production of the X-ray images, as a result of which it is possible to generate an X-ray field which is not arranged symmetrically with respect to the center or middle of the detector area. The edge zones of the receiving area may be used, for example, to produce images without the patient being required to be repositioned or to assume an uncomfortable position on or at the patient examination table.

The radiation generator unit and the receiving area of the image receiver can be moved relative to one another in such a way that the receiving area of the image receiver is masked out asymmetrically with respect to image receiver center and/or the X-ray field is generated asymmetrically with respect to the center of the receiving area of the image receiver. This allows optimal use of the available detector area.

The radiation generator unit and the receiving area of the image receiver can be moved relative to one another by rotating the radiation generator unit through an angle relative to the receiving area of the image receiver and/or by rotating the receiving area of the image receiver through an angle relative to the radiation generator unit.

The radiation generator unit may be rotated or pivoted while the image receiver area remains stationary. Rotating the radiation generator unit allows that edge zones of the receiving area to be optimally illuminated without the generated X-ray field having to be chosen unnecessarily large for the desired image acquisition.

The radiation generator unit and the receiving area of the image receiver may be moved relative to one another by displacing the radiation generator unit relative to the receiving area of the image receiver, for example, by displacing it parallel to the image receiver plane, and/or by displacing the receiving area of the image receiver relative to the radiation generator unit.

The displacement is usually realized as a displacing of the radiation generator unit, preferably, but not necessarily, represents an alternative to the rotating in particular of the emitter or of the plurality of emitters of the radiation generator unit. The central beam of the X-ray field may be masked-in onto the receiving area strikes at right angles onto the object even after the displacing and strikes at right angles onto the image receiver, with the result that there is no risk of image distortions due to an oblique or inclined striking of the beam.

The radiation generator unit can be assigned a grid unit, in particular a symmetrically structured grid unit, for generating radiation. Symmetric grid units, in which the grid pairs are moved (exclusively) symmetrically with respect to the central beam, are comparatively inexpensive compared to asymmetric grids. Commercially available multi-leaf collimators can be used for generating sub-fields of the solid-state detector marginally in the longitudinal axis of the patient for irradiation purposes in order thereby to achieve a reduction in the overall radiation dose applied.

Accordingly there is no need to choose an unnecessarily large X-ray field, while at the same time one grid in which the grid pairs are in each case moved only symmetrically is sufficient for acquiring optimal images.

To allow the relative movement of the radiation generator unit and of the image receiver with respect to one another, a movement coupling device, such as a mechanical and/or electromechanical movement coupling device, can be released. An image acquisition may be performed with a fixed coupling of the radiation generator unit with respect to the image receiver in order thereby to produce unproblematic images. Only in the case where images have to be taken in an end position of a movement axis of the X-ray machine, or possibly in further exceptional cases, is this coupling, which can be based on different active principles, released. A desired relative movement between the emitter and the receiving area is then possible.

After the releasing of the movement coupling device the radiation generator unit and/or the receiving area of the image receiver can be moved and/or guided manually and/or in a motor-driven manner relative to one another, for example, by moving the radiation generator unit parallel to the longitudinal axis of a positioning device of the X-ray device. This movement of the radiation generator unit and the receiving area of the image receiver relative to one another can be implemented continuously and/or incrementally.

### BRIEF DESCRIPTION OF THE DRAWINGS

Further advantages, features and details of the method will emerge on the basis of the following exemplary embodiments as well as with reference to the drawings, in which:

FIG. 1 is a schematic representation illustrating a symmetrical illumination of a receiving area of an image receiver,

FIGS. 2 and 3 are schematic representations illustrating asymmetrical masking-out by rotating a radiation generator unit,

FIGS. 4 and 5 are schematic representations illustrating asymmetrical masking-out by displacing the radiation generator unit parallel to the imaging plane,

FIG. 6 shows an X-ray device having a displaceable radiation generator unit, and

FIG. 7 shows an example illustrating the use of a method.

### DETAILED DESCRIPTION

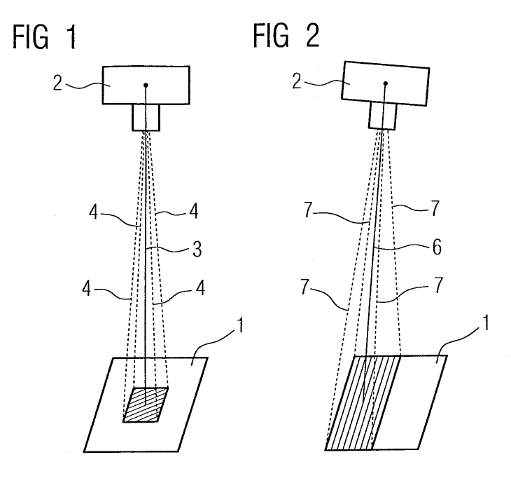

FIG. 1 shows a symmetrical illumination of a receiving area 1 of an image receiver. An X-ray field that is considerably smaller compared to the overall receiving area 1 of the detector is generated by a radiation generator unit 2. The X-ray field has a central beam 3, which is directed precisely onto the center of the receiving area 1. The edge beams 4 are arranged symmetrically with respect to the center of the receiving area 1. The radiation field is structured symmetrically with respect to the central beam.

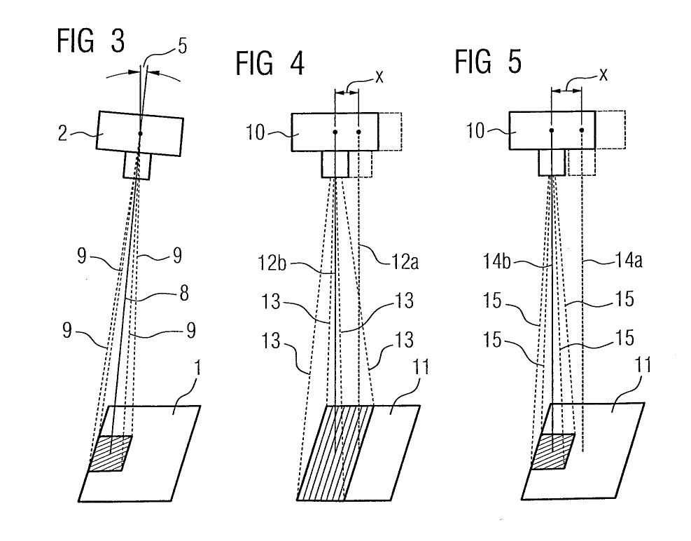

FIGS. 2 and 3 show asymmetric masking-out and illumination of the receiving area 1. The asymmetric masking-out and irradiation of the receiving area 1 of the detector are made possible as a result of the fact that the radiation generator unit 2 is rotated through a specific angle, identified in FIG. 3 by the reference sign 5, in the direction of the displacement of the X-ray device and hence also in the direction toward the edge zone of the receiving area 1.

FIG. 2 shows the case where the entire length of the receiving area 1 (into the representation plane of FIG. 2) is used.

The central beam 6 around which the edge beams 7 are in turn symmetrically arranged do not strike the center of the receiving area 1, but strikes the receiving area 1 eccentrically or non-centrally. In the example shown in FIG. 2, roughly half the width of the receiving area 1 (from left to right in the representation of FIG. 2) and the entire length of the receiving area 1 are illuminated.

In the example shown in FIG. 3, the central beam 8 strikes the receiving area eccentrically, the edge beams 9 being arranged in this case in such a way that the entire length of the receiving area 1 is also not used. A small area in the image may be acquired in a targeted manner if necessary, without the patient for whom the image is to be produced being exposed to an unnecessarily high dose of radiation.

FIGS. 4 and 5 show representations illustrating asymmetric masking-out by parallel displacement of a radiation generator unit 10 relative to the imaging plane, or example, to the receiving area 11. The parallel displacement capability usually represents an alternative to the rotating of a radiation generator unit, so that in one X-ray device only one or the other solution will be implemented. However, both a parallel displacing of the emitter and a rotating of the emitter will be possible in one and the same X-ray device.

FIG. 4 shows the radiation generator unit 10 being displaced starting from its normal position through a quarter of the detector width (i.e. in the longitudinal direction of a patient positioning device or in this case to the left in the diagram). At the same time a masking-in is performed in such a way that half of the receiving area 11 is used. For example, such that after the displacing compared to the previous central beam 12a the central beam 12b is surrounded by edge beams 13, two of which lie on corners of the receiving area 11, while the other two strike the receiving area 11 on the centers of the side edges extending in each case from the corners in the direction of the detector width.

In FIG. 5, as a result of the displacing of the radiation generator unit 10 whose previous position is indicated, as shown in FIG. 4, by a dashed outline, the previously centrally striking central beam 14a is mapped onto a new central beam 14b which now no longer strikes the center of the receiving area 11, but is arranged eccentrically and surrounded by edge beams 15, two of which strike an edge of the receiving area 11, while the other two strike the receiving area 11 in an inner area. The radiation generator unit 10 is displaced through a specific path, designated here as x.

This solution of FIGS. 4 and 5 offers the advantage that the receiving area 11 is struck at right angles, thereby preventing image distortions.

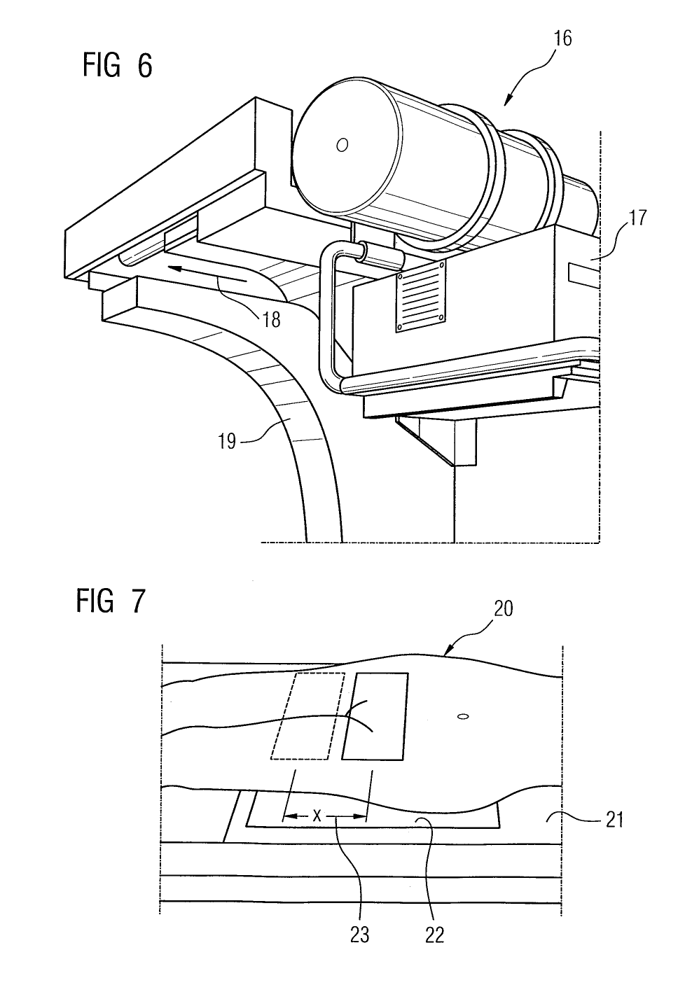

FIG. 6 lastly shows an X-ray device 16, which in this case is depicted only partially for reasons of clarity, having a displaceable radiation generator unit 17. The X-ray device 16 is a urological X-ray machine in which according to the invention a mechanical adjustment of the position of the radiation generator unit 17 in the direction of the longitudinal axis relative to the detector is provided for the purpose of implementing an asymmetric masking-in of radiation onto a detector field that is not shown in this case.

In other exemplary embodiments an electromechanical displacement means can likewise be provided alternatively or in addition.

The radiation generator unit 17 is thus, as indicated here by the arrow 18, movable longitudinally with respect to the detector area, an asymmetric radiation collimation being made possible for the purpose of producing images in end positions of the movement axis of the machine, i.e. In this case the X-ray device 16, as a result of the releasing of the coupling of the movement of the radiation generator unit 17 to the movement of the detector area.

In the case shown here a continuous adjustment is possible in the direction indicated by the arrow 18. Equally, however, incremental adjustment options can also be provided in other exemplary embodiments.

In this case, therefore, the radiation generator unit 17 is moved in the direction of the arrow 18 relative to a mount 19 with respect to which the position of the receiving area of the detector is in turn fixed. Thus, as a result of the movement of the radiation generator unit 17 a relative movement is achieved between the radiation generator unit 17 and the receiving area in the form of a parallel displacement of the radiation generator unit 17 relative to the receiving area.

FIG. 7 shows an example intended to illustrate the use of the method according to the invention, wherein in this case an image of the urinary tract or bladder of a patient 20, represented only schematically here, is to be generated. For this purpose the patient 20 is positioned on a patient support 21 in which the image receiver 22 is also disposed.

With prior art X-ray devices it is necessary for the object or the region to be imaged to be brought into the center of the image receiver 22. This means that in the case shown here the pelvis would have to be repositioned or longitudinally displaced relative to an initial examination position. However, this is associated with a certain amount of effort or even pain for the patient, while the duration of the examination is also increased. Furthermore it is possible due to the repositioning that the comparability of images produced in different rest positions will be compromised.

Consequently, according to the invention a radiation generator unit (not shown here) is displaced through a path x, as indicated by the double arrow 23, if necessary so that the desired image can be produced without a repositioning of the patient 20 by masking out or, as the case may be, irradiating the image receiver 22 asymmetrically with respect to its center.

No specific asymmetric grids are required for this, but instead inexpensive symmetric multileaf collimators can be used, with the result that the method according to the invention can be implemented in the most disparate applications without major effort or expense.

1. An X-ray device for producing X-ray images in an end position of at least one movement axis of the X-ray device, the X-ray device comprising:
a radiation generator unit that generates an X-ray field, and
an image receiver that has a larger receiving area compared to an area of the X-ray field,
wherein the radiation generator unit and the receiving area of the image receiver are movable relative to one another.

2. The X-ray device as claimed in claim 1, wherein the radiation generator unit and the receiving area of the image receiver are movable relative to one another in such a way that the receiving area of the image receiver are masked out asymmetrically with respect to a center and/or the X-ray field that is to be generated or has been generated is situated asymmetrically with respect to the center of the receiving area of the image receiver.

3. The X-ray device as claimed in claim 1, wherein the radiation generator unit and the receiving area of the image receiver are movable relative to one another by rotating the radiation generator unit through an angle relative to the receiving area of the image receiver and/or by rotating the receiving area of the image receiver through an angle relative to the radiation generator unit.

4. The X-ray device as claimed in claim 1, wherein the radiation generator unit and the receiving area of the image receiver are movable relative to one another by displacing the radiation generator unit relative to the receiving area of the image receiver by displacing the radiation generator parallel to the image receiver plane and/or by displacing the receiving area of the image receiver relative to the radiation generator unit.

5. The X-ray device as claimed in claim 1, wherein the image receiver is a solid-state detector.

6. The X-ray device as claimed in claim 1, wherein the radiation generator unit is assigned a symmetrically structured grid unit for generating radiation.

7. The X-ray device as claimed in claim 6, wherein the structured grid unit includes grid pairs that are movable symmetrically with respect to the central beam.

8. The X-ray device as claimed in claim 1, wherein the radiation generator unit and the receiving area of the image receiver are coupled to enable a relative movement by a releasable movement coupling device.

9. The X-ray device as claimed in claim 8, wherein a mechanical and/or electromechanical movement coupling device are provided as the releasable movement coupling device.

10. The X-ray device as claimed in claim 9, wherein after the releasing of the movement coupling device the radiation generator unit and/or the receiving area of the image receiver can be moved and/or guided relative to one another manually and/or in a motor-driven manner by moving the radiation generator unit parallel to the longitudinal axis of a positioning device of the X-ray device.

11. The X-ray device as claimed in claim 1, wherein the radiation generator unit and the receiving area of the image receiver are operable to be moved relative to one another continuously and/or incrementally.

12. A method for producing X-ray images in an end position of at least one movement axis of an X-ray device, the method comprising:
generating an X-ray field using a radiation generator unit, the X-ray field being smaller than a receiving area of an image receiver,
moving the radiation generator unit and the receiving area of the image receiver relative to one another when generating the X-ray field,
wherein the X-ray field is used to produce X-ray images.

13. The method as claimed in claim 12, comprising moving the radiation generator unit and the receiving area of the image receiver relative to one another in such a way that the receiving area of the image receiver are masked out asymmetrically with respect to its center and/or the X-ray field is generated asymmetrically with respect to the center of the receiving area of the image receiver.

14. The method as claimed in claim 12, comprising moving the radiation generator unit and the receiving area of the image receiver relative to one another by rotating the radiation generator unit through an angle relative to the receiving area of the image receiver and/or by rotating the receiving area of the image receiver through an angle relative to the radiation generator unit.

15. The method as claimed in claim 12, comprising moving the radiation generator unit and the receiving area of the image receiver relative to one another by displacing the radiation generator unit relative to the receiving area of the image receiver by displacing it parallel to the image receiver plane, and/or by displacing the receiving area of the image receiver relative to the radiation generator unit.

16. The method as claimed in claim 12, generating the radiation using a grid unit assigned to the radiation generator unit, the grid unit including grid pairs that are movable symmetrically with respect to a central beam of the X-ray field.

17. The method as claimed in claim 12, comprising releasing a movement coupling device to allow the movement of the radiation generator unit and the receiving area of the image receiver relative to one another.

18. The method as claimed in claim 12, wherein after the releasing of the movement coupling device the radiation generator unit and/or the receiving area of the image receiver are moved and/or guided relative to one another manually and/or in a motor-driven manner, in particular by moving the radiation generator unit parallel to the longitudinal axis of a positioning device of the X-ray device.

19. The method as claimed in claim 12, comprising moving the radiation generator unit and the receiving area of the image receiver relative to one another continuously and/or incrementally.

20. The method as claimed in claim 17, wherein the movement coupling device is a mechanical and/or electromechanical movement coupling device.
